(19) United States
(12) Patent Application Publication
(10) Pub. No.: US 2022/0000679 A1
(43) Pub. Date: Jan. 6, 2022

(54) HYGIENIC PAD

(71) Applicants: Jea Won LEE, Seoul (KR); J2LFA Co., Ltd, Seoul (KR)

(72) Inventors: Woo-Bok LEE, Seoul (KR); Jea Won LEE, Seoul (KR)

(73) Assignees: J2LFA Co., Ltd, Seoul (KR); Jea Won LEE, Seoul (KR)

(21) Appl. No.: 16/918,583

(22) Filed: Jul. 1, 2020

(51) Int. Cl. A61F 13/475; A61F 13/56; A61F 13/515; A61F 13/511

(52) U.S. Cl. CPC A61F 13/4758; A61F 13/5616; A61F 2013/4518; A61F 13/5116; A61F 13/515

(57) ABSTRACT

The sanitary pad of the present invention includes: (i) a lower cover layer 10 including a straight type body 11 and wing parts 12; (ii) an absorbent pad layer 20 whose both sides are separated from the lower cover layer 10 and only central portion is adhered to the lower cover layer 10, wherein a distal end portion thereof is divided into two hemispherical protrusions 21 and 22; (iii) an upper cover layer 30 laminated on the absorbent pad layer 20; and (iv) side cover layers 40 whose both ends extend from upper edges on both sides of the upper cover layer 30 to both upper end portions of the cover layer 10 in the lateral direction thereof, and middle portions are inserted into a gap formed between the lower cover layer 10 and the separated both side portions of the absorbent pad layer 20.

### BACKGROUND OF THE INVENTION

### 1. Field of the Invention

The present invention relates to a sanitary pad, and more specifically, to a sanitary pad that can maintain a state in which both side parts of an absorbent pad layer 20 separated from a lower cover layer 10 are in close contact with a human body, even if the lower cover layer 10 attached to underwear is moved away from the human body due to a movement of a wearer of the sanitary pad, and can reliably come into close contact with a crotch portion of the wearer to effectively prevent leakage of menstrual blood.

### 2. Description of the Related Art

As a conventional sanitary pad capable of containing menstrual blood, as shown in FIGS. 1 and 2, a sanitary pad including: (i) a lower cover layer 10 made of a film material; (ii) an absorbent pad layer 20 adhered to a central portion of the lower cover layer 10 in a longitudinal direction of the lower cover layer 10; and (iii) an upper cover layer 30 covering the absorbent pad layer 20 and the lower cover layer 10 has been mainly used.

The absorbent pad layer 20 is made of a non-woven material or a cotton pulp material, etc., and the upper cover layer 30 is made of a non-woven material or a mesh film material, etc.

The conventional sanitary pad is described in Korean Utility Model Publication No. 2019-684 and Korean Utility Model Registration Publication No. 20-305833.

However, in a case of the conventional sanitary pad, an entire lower area of the absorbent pad layer 20 is integrally adhered to the lower cover layer 10, and an entire lower area of the upper cover layer 30 is integrally adhered to the absorbent pad layer 20 and the lower cover layer 10, thereby the absorbent pad layer 20 and the upper cover layer 30 are forced to move together without being separated from the lower cover layer 10. Therefore, when the lower cover layer 10 of the sanitary pad adhered to the underwear is moved away from the human body due to a movement of a wearer of the sanitary pad, the absorbent pad layer 20 and the upper cover layer 30 are also naturally moved away from the human body and do not come into close contact with the human body as well as a crotch portion of the wearer, such that a problem of leakage of menstrual blood occurs.

### SUMMARY OF THE INVENTION

It is an object of the present invention to provide a sanitary pad that can maintain a state in which both side parts of an absorbent pad layer 20 separated from a lower cover layer 10 are in close contact with a human body, even if the lower cover layer 10 attached to underwear is moved away from the human body due to a movement of a wearer of the sanitary pad, and can reliably come into close contact with a crotch portion of the wearer to effectively prevent leakage of menstrual blood.

In order to achieve the above-described object, according to an aspect of the present invention, as shown in FIGS. 3 and 4, when manufacturing a sanitary pad by sequentially laminating a lower cover layer 10 made of a film material, an absorbent pad layer 20, side cover layers 40 and an upper cover layer 30, both sides of the absorbent pad layer 20 are separated from the lower cover layer 10, and only a central portion thereof is adhered to the lower cover layer 10, and the side cover layers 40 extend from upper edges on both sides of the upper cover layer 30 to both end portions of the cover layer 10, and then middle portions of the side cover layers 40 are inserted into a gap formed between the lower cover layer 10 and the separated both side portions of the absorbent pad layer 20 among (i) the lower cover layer 10 and (ii) the absorbent pad layer 20.

Further, in the present invention, a distal end portion of the absorbent pad layer 20 is formed to be divided into two hemispherical protrusions 21 and 22.

According to the present invention, both side portions of the absorbent pad layer 20 are separated from the lower cover layer 10, and then the separated both side portions are wrapped with the side cover layers 40. Therefore, even if the lower cover layer 10 attached to the underwear is moved away from a human body due to a movement of a wearer of the sanitary pad, the absorbent pad layer 20 reliably comes into close contact with a crotch portion of the wearer by increasing a contact area with the human body. In addition, the distal end portion of the absorbent pad layer 20 is divided into two hemispherical protrusions 21 and 22, and the lower end portion of the sanitary pad is formed in a form that is folded depending on a difference in the thickness, thereby it is possible to effectively prevent the leakage of menstrual blood by increasing a degree of adhesion of the sanitary pad in accordance with a curved surface of the human body.

### BRIEF DESCRIPTION OF THE DRAWINGS

The above and other objects, features and other advantages of the present invention will be more clearly understood from the following detailed description taken in conjunction with the accompanying drawings, in which:

FIG. 1 is a perspective view of a conventional sanitary pad;

FIG. 2 is an enlarged cross-sectional view taken on line A-A of FIG. 1;

FIG. 3 is a perspective view of an assembled sanitary pad according to the present invention; and

FIG. 4 is an enlarged cross-sectional view taken on line B-B of FIG. 3.

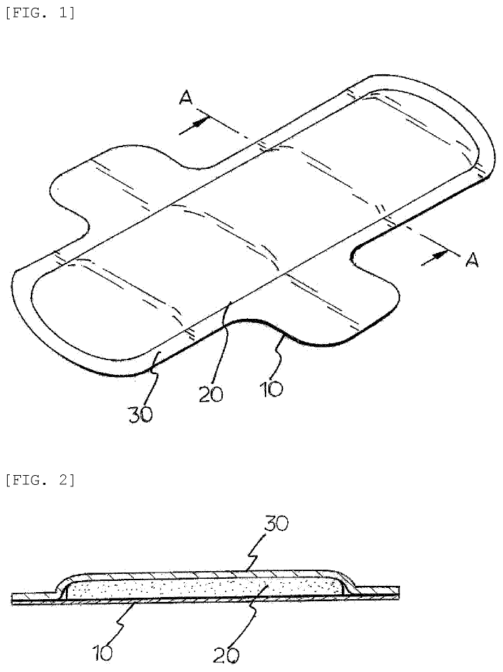

### DETAILED DESCRIPTION OF THE INVENTION

Hereinafter, the present invention will be described in detail with reference to the accompanying drawings.

The sanitary pad according to the present invention is characterized by including: (i) a lower cover layer 10 which includes a straight type body 11 and wing parts 12 formed at respective center portions on both sides of the straight type body 11 in a lateral direction thereof, and is made of a film material; (ii) an absorbent pad layer 20 whose both sides are separated from the lower cover layer 10 at a central portion of the lower cover layer 10 in a longitudinal direction of the lower cover layer 10 and only central portion is adhered to the lower cover layer 10, wherein a distal end portion thereof is divided into two hemispherical protrusions 21 and 22; (iii) an upper cover layer 30 laminated on the absorbent pad layer 20; and (iv) side cover layers 40 having a form in which both ends thereof extend from upper edges on both sides of the upper cover layer 30 to both upper end portions of the cover layer in the lateral direction thereof, and middle portions thereof are inserted into a gap formed between the lower cover layer 10 and the separated both side portions of the absorbent pad layer 20 among the lower cover layer 10 and the absorbent pad layer 20.

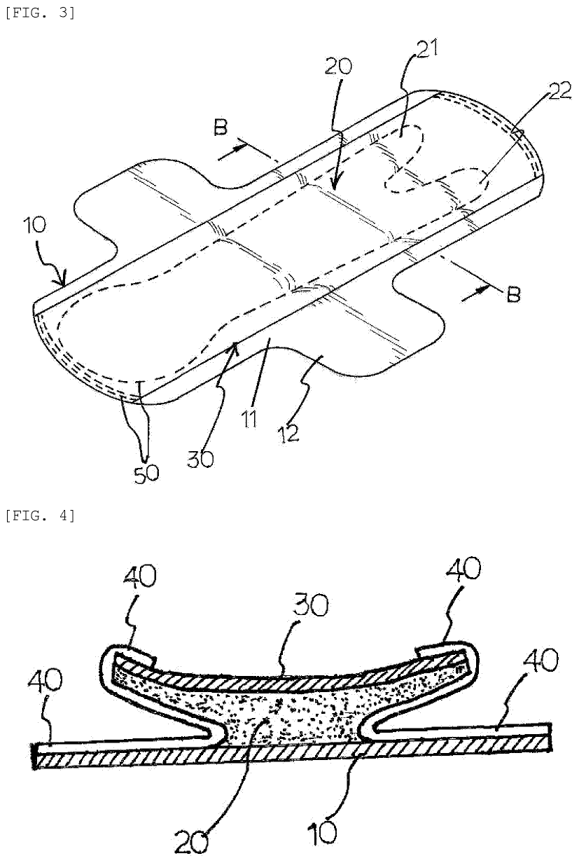

Specifically, as shown in FIG. 3, the lower cover layer 10 forming the sanitary pad of the present invention includes the straight type body 11 and the wing parts 12 formed at the respective center portions on both sides of the straight type body 11 in the lateral direction thereof, and is made of a film material.

In addition, as shown in FIG. 4, the absorbent pad layer 20 forming the sanitary pad of the present invention has a configuration in which both sides are separated from the lower cover layer 10 at the central portion of the lower cover layer 10 in the longitudinal direction of the lower cover layer 10 and only the central portion is adhered to the lower cover layer 10, and is made of a non-woven material or a cotton pulp material, etc.

The absorbent pad layer 20 is characterized in that the distal end portion thereof is divided into the two hemispherical protrusions 21 and 22 so that the absorbent pad layer 20 reliably comes into close contact with a crotch portion of the human body of the wearer. In addition, a proximal end portion of the absorbent pad layer 20 is adhered to the lower cover layer 10 along an adhesion line 50. In the present disclosure, the “distal end” refers to a side which comes into close contact with the crotch portion (the right side in FIG. 3), and the “proximal end” refers to a side opposite thereto (the left side in FIG. 3).

The upper cover layer 30 forming the sanitary pad of the present invention is laminated on the absorbent pad layer 20, and is made of a non-woven material or a mesh film material, etc.

The side cover layers 40 forming the sanitary pad of the present invention are made of a non-woven material or a mesh film material, and have a form in which the both ends thereof extend from the upper edges on both sides of the upper cover layer 30 to the both upper end portions of the cover layer 10 in the lateral direction thereof, and the middle portions thereof are inserted into the gap formed between the lower cover layer 10 and the separated both side portions of the absorbent pad layer 20 among the lower cover layer 10 and the absorbent pad layer 20.

The present invention is characterized in that the both side portions of the absorbent pad layer 20 wrapped with the side cover layers 40 are separated from the lower cover layer 10.

The both side portions of the absorbent pad layer separated from the lower cover layer 10 play a role of a barrier membrane to minimize the gap between the sanitary pad and the human body, thus to prevent the menstrual blood leaking to an outside by increasing the contact area with the human body, even if the lower cover layer 10 attached to the underwear is moved away from the human body due to a movement of the wearer of the sanitary pad.

According to the present invention, both side portions of the absorbent pad layer 20 are separated from the lower cover layer 10, and then the separated both side portions are wrapped with the side cover layers 40. Therefore, even if the lower cover layer 10 attached to the underwear is moved away from a human body due to a movement of a wearer of the sanitary pad, the absorbent pad layer 20 reliably comes into close contact with a crotch portion of the wearer by increasing a contact area with the human body. In addition, the distal end portion of the absorbent pad layer 20 is divided into two hemispherical protrusions 21 and 22, and the lower end portion of the sanitary pad is formed in a form that is folded depending on a difference in the thickness, thereby it is possible to effectively prevent the leakage of menstrual blood by increasing a degree of adhesion of the sanitary pad in accordance with a curved surface of the human body.

### Description of Reference Numerals

10 Lower cover layer

11 Straight type body of lower cover layer

12 Wing part of lower cover layer

20 Absorbent pad layer

21, 22 Hemispherical protrusion of absorbent pad layer

30 Upper cover layer

40 Side cover layer

50 Adhesion line

What is claimed is:

1. A sanitary pad comprising:
(i) a lower cover layer 10 which includes a straight type body 11 and wing parts 12 formed at respective center portions on both sides of the straight type body 11 in a lateral direction thereof, and is made of a film material;
(ii) an absorbent pad layer 20 whose both sides are separated from the lower cover layer 10 at a central portion of the lower cover layer 10 in a longitudinal direction of the lower cover layer 10 and only central portion is adhered to the lower cover layer 10, wherein a distal end portion thereof is divided into two hemispherical protrusions 21 and 22;
(iii) an upper cover layer 30 laminated on the absorbent pad layer 20; and
(iv) side cover layers 40 having a form in which both ends thereof extend from upper edges on both sides of the upper cover layer 30 to both upper end portions of the cover layer 10 in the lateral direction thereof, and middle portions thereof are inserted into a gap formed between the lower cover layer 10 and the separated both side portions of the absorbent pad layer 20 among the lower cover layer 10 and the absorbent pad layer 20.
